(19) United States
(12) Patent Application Publication
(10) Pub. No.: US 2023/0398284 A1
(43) Pub. Date: Dec. 14, 2023

(54) DEVICES, SYSTEMS, AND METHODS FOR DELIVERING FLUID THROUGH A TUBULAR ELEMENT

(71) Applicants: BOSTON SCIENTIFIC SCIMED, INC., Maple Grove, MN (US); MAYO FOUNDATION FOR MEDICAL EDUCATION AND RESEARCH, Rochester, MN (US)

(72) Inventors: Jason John Matteson, JR., Beldenville, WI (US); Matthew Maciej, Rogers, MN (US); Mitchell Nelson, Hudson, WI (US); Paul A. Friedman, Rochester, MN (US)

(73) Assignees: BOSTON SCIENTIFIC SCIMED, INC., Maple Grove, MN (US); MAYO FOUNDATION FOR MEDICAL EDUCATION AND RESEARCH, Rochester, MN (US)

(21) Appl. No.: 18/201,562

(22) Filed: May 24, 2023

Related U.S. Application Data
(60) Provisional application No. 63/345,572, filed on May 25, 2022.

(51) Int. Cl. A61M 5/00; F16L 11/08; A61B 17/04

(52) U.S. Cl. CPC A61M 5/007; A61B 17/0401; F16L 11/085

(57) ABSTRACT

A flexible elongate member with the ability to deliver a material through a medium-delivery channel defined within a wall thereof. The flexible elongate member may have a tubular wall defining a lumen therethrough, the medium-delivery channel being formed within the tubular wall. The flexible elongate member may be a reinforced flexible tubular elongate member with a reinforcing layer within the tubular wall thereof. One or more filaments forming the reinforcing layer may be hollow to define the medium-delivery channel extending through the reinforced flexible tubular elongate member.

### CROSS-REFERENCE TO RELATED APPLICATIONS

This application claims the benefit of priority under 35 U.S.C. § 119 to U.S. Provisional Application No. 63/345,572, filed May 25, 2022, the entire disclosure of which is hereby incorporated by reference herein for all purposes.

### FIELD

The present disclosure relates generally to the field of medical devices. More particularly, the present disclosure relates to medical devices, systems, and methods for delivering and/or deploying devices to an anatomical site, and also delivering additional material, such as a fluid medium, to the anatomical site. Even more particularly, the present disclosure relates to medical devices, systems, and methods for delivering and deploying an implantable device as well as delivering a material, such as a contrast agent, with the implantable device to facilitate implanting of the device.

### BACKGROUND

Devices, systems, and methods for delivering and/or deploying devices with minimally invasive techniques, such as transluminally, are desirable for avoiding more complex and invasive open surgery procedures. Various transluminal techniques, which do not require open surgery, utilize systems with flexible elongate members capable of navigating to an anatomical site within the body from a small insertion opening in a patient's body transluminally through the body, such as through the vascular system, to an anatomical site. Such systems may be multi-catheter/stacked catheter systems which include a plurality of flexible tubular elongate members stacked one within the other (coaxially and/or coextensively within another flexible tubular elongate member). It may be desirable to deliver additional materials with such systems. For instance, to deliver and/or deploy a device, it may be desirable to use an imaging system which utilizes delivery of contrast agents to the anatomical site. However, transluminally-delivered devices, systems, etc., typically face strict size constraints to navigate effectively within the body. Therefore, there generally is limited to no room or space within multi-catheter systems for an additional channel or passage for delivering materials to (or removing materials from) an anatomical site. Devices, systems, therapies, etc., which utilize more than one flexible elongate member may thus have limited ability to be able to deliver additional materials to an anatomical site through an already maximum-sized system. Solutions to such challenges would be welcome in the industry.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a flexible tubular elongate member has a tubular wall defining a lumen through which a medical device is deliverable to an anatomical site, the flexible tubular elongate member comprising at least one hollow filament extending within the tubular wall, from a first end of said flexible tubular elongate member to a second end of the flexible tubular elongate member, and defining a medium-delivery channel therein through which a material may pass within the tubular wall from the first end of the flexible tubular elongate member to the second end of the flexible tubular elongate member.

In some embodiments, the tubular wall is a multi-layer wall. In some embodiments, tubular wall has an outer layer and an inner layer, with the hollow filament extending therebetween longitudinally along the flexible tubular elongate member. In some embodiments, the hollow filament is woven with a plurality of reinforcement filaments to form a reinforcing layer between the outer layer and the inner layer of the flexible tubular elongate member. In some embodiments, more than one of the plurality of reinforcement filaments are hollow, extend from a first end of the flexible tubular elongate member to a second end of the flexible tubular elongate member, and define a medium-delivery channel therein through which a material may pass from the first end of the flexible tubular elongate member to the second end of the flexible tubular elongate member.

In some embodiments, the hollow filament is embedded within the material of the tubular wall.

In some embodiments, the hollow filament is one of a plurality of filaments extending longitudinally along the flexible tubular elongate member and within the tubular wall. In some embodiments, the plurality of filaments form a reinforcing layer within the tubular wall. In some embodiments, the hollow filament is one of the plurality of hollow filaments forming the reinforcing layer.

In accordance with various principles of the present disclosure, a delivery and/or deployment system, for delivering and/or deploying a medical device to an anatomical site, comprises at least one flexible tubular elongate member having a wall defining a lumen and a medium-delivery channel defined within the wall and extending longitudinally along the flexible tubular elongate member between a proximal end thereof and a distal end thereof; and a device delivered at the distal end of the flexible tubular elongate member.

In some embodiments, the device comprises a delivery device having an interior within which a deployable device is positioned. In some embodiments, the lumen defined by the flexible tubular elongate member wall is in fluid communication with the interior of the delivery device. In some embodiments, the deployable device is a tissue anchor. In some embodiments, the tissue anchor is biased to shift from a deployment configuration to a deployed configuration upon exiting an open distal end of the delivery device. In some embodiments, a medium is deliverable through the medium-delivery channel within the wall of the flexible tubular elongate member, into the interior of the delivery device, and out the open distal end of the delivery device.

In some embodiments, the medium-delivery channel is formed within a hollow reinforcement filament extending within the wall of the flexible tubular elongate member between the proximal end and the distal end of the flexible tubular elongate member.

In accordance with various principles of the present disclosure, a method of delivering a material to a distal end of a reinforced flexible tubular elongate member comprises delivering a material through a medium-delivery channel defined longitudinally along a hollow reinforcement filament extending within the wall of the reinforced flexible tubular elongate member between a proximal end and a distal end of the reinforced flexible tubular elongate member.

In some embodiments, the method further includes delivering a material through a plurality of hollow reinforcement filaments braided to form a reinforcing layer within the wall of the reinforced flexible tubular elongate member.

In some embodiments, the method further includes delivering a contrast agent to the distal end of the reinforced flexible tubular elongate member through the hollow reinforcement filament to determine the position of the reinforced flexible tubular elongate member with respect to tissue. In some embodiments, the method further includes confirming the position of the distal end of the reinforced flexible tubular elongate member with respect to tissue at an anatomical site, and implanting an implantable device delivered at the distal end of the reinforced flexible tubular elongate member into the tissue. These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a flexible tubular elongate member having a medium-delivery channel defined within a wall thereof in accordance with various principles of the present disclosure.

FIG. 2 illustrates a perspective view of an example of an embodiment of a device and system formed in accordance with various principles of the present disclosure shown in a schematic representation of a heart.

FIG. 3 illustrates a further perspective view of an example of an embodiment of a device and system such as illustrated in FIG. 2, in a further advanced position.

FIG. 4 illustrates a cross-sectional view along line IV-IV of FIG. 2, but with the deployable device, illustrated in FIG. 2 in a delivery configuration within a delivery device, extended out of the delivery device.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore.

Traditionally, medical devices are delivered and/or deployed transluminally (within the body without the need for an open surgical procedure) with systems including a variety of flexible elongate members. For instance, a medical device may be inserted through a guide catheter (e.g., of a delivery/deployment system) and/or along or with the use of one or more flexible elongate members. It will be appreciated that the term flexible elongate member or flexible tubular elongate member is used herein generically to refer to elements such as catheters, shafts, cannulas, sheaths, tubes, stylets, endoscopes, etc., for the sake of convenience and without intent to limit. Generally, devices and/or systems are navigated to a site within a patient's body such as an implant/implantation site, a target site, a delivery site, a deployment site, a treatment site, etc., hereinafter referenced simply as an anatomical site for the sake of convenience and without intent to limit. The device may be associated with tissue at the anatomical site, such as by being attached, anchored, implanted, affixed, secured, coupled, engaged, embedded, held, retained, purchased, secured, etc., with or with respect to tissue, such terms (and various grammatical forms thereof) being used interchangeably herein without intent to limit. It will be appreciated that references herein to delivery and/or deployment are intended to include delivery, or deployment, or both. It will further be appreciated that deployment may include securing in addition to placing of a device. The delivery/deployment device may be configured to hold and/or carry a device for delivery to the deployment site, and/or to facilitate deployment of the device with respect to tissue. For instance, the delivery/deployment device may be configured to manipulate the device for positioning and deploying (e.g., implanting) with respect to tissue at an anatomical site. It will be appreciated that terms such as navigate (and other grammatical forms thereof) may be used interchangeably herein with such terms (and other grammatical forms thereof) as navigate, actuate, control, maneuver, manipulate, move, operate, shift, transition, drive, advance, retract, rotate, translate, etc., without intent to limit.

Devices and systems are often delivered transluminally in a multi-component delivery system, with two, three, four, or more flexible elongate members positioned one within the other (coaxially, concentrically, or otherwise one within another and/or alongside one another and optionally also within another tubular elongate members) in a configuration known as a catheter stack-up delivery system. The flexible elongate members may include outer shafts, delivery shafts, catheters, sheaths, stylets, guidewires, tubes, etc., reference being made herein to flexible elongate members for the sake of convenience and without intent to limit. Various of the flexible elongate members may be flexible tubular elongate members made of a plurality of components or layers, such as two, three, or more tubular layers. The use of multiple different layers may allow for providing a flexible tubular elongate member with a variety of characteristics. For instance, one or more multiple-layer flexible tubular elongate members may include an inner layer (alternately referenced herein as a liner without intent to limit) made of a lubricious material to facilitate insertion and transport of medical devices therein and therethrough. Such inner layer may be formed of a material such as, but not limited to, polytetrafluoroethylene (PTFE), tetrafluoroethylene with perfluoroalkyl vinyl ether (PFA), perfluoropropyl vinyl ether, perfluoromethyl vinyl ether, fluorinated ethylene propylene (FEP), polyether block amide (PEBA), or copolymers or blends thereof. The inner layer may be formed in a variety of manners, such as an extrusion process, or by molding or coating a material over a mandrel or within another layer (a relatively outer layer) of the flexible tubular elongate member. In some embodiments, an outer layer (which may alternatively be referenced as a jacket without intent to limit) of a multi-layer flexible tubular elongate member may be formed of a generally lubricious material providing the flexible tubular elongate member with a frictionless (or at least reduced friction) biocompatible outer surface to facilitate maneuvering of the flexible tubular elongate member within passageways/lumens of a patient's body without damaging surrounding tissue. Such outer layer may be formed of any of a variety of biocompatible materials, such as nylon, polyurethane, polyether block amide (PEBA), PTFE, high density polyethylene (HDPE), liquid crystal polymer (LCP), or copolymers or blends thereof, in any of a desired manners, such as extrusion, dipping, painting, brush coating, spraying, molding, etc., the coating material over the other layers of the flexible tubular elongate member. It will be appreciated that the flexible tubular elongate member may have differing characteristics (e.g., flexibility, stiffness, wall thickness, elasticity, durometer, etc.) along the length thereof.

Various devices and systems as described above may utilize reinforced flexible tubular elongate members such as to insert, guide, deliver, deploy, etc., a medical device transluminally within a patient's body. Reinforcement of a flexible elongate member may be desirable to impart one or more properties or characteristics to the flexible elongate member, such as strength, flexibility (or a desired degree of stiffness), enhanced maneuverability, tensile strength, torque transmission abilities, steering capabilities, stability, durability, elasticity, resistance to fracture, etc., such as to give the flexible elongate member various abilities needed for navigating within a complex system such as within the body, delivering a device or system or treatment or therapy, implanting a device or system, manipulating a device or system, etc., or otherwise selected for an intended use of the reinforced flexible tubular elongate member It will be appreciated that not all properties or characteristics of the reinforcing layer may be considered to “reinforce”, and reference to and use of the term reinforce, and other grammatical forms thereof, herein is for the sake of convenience and not necessarily limited to traditional concepts of “reinforcing.”

The reinforcing layer of a multi-layer flexible tubular elongate member may be formed over the inner layer, or formed independently with the material of the inner layer applied thereto to form the inner layer. In some embodiments, the reinforcing layer may be embedded into the inner layer. In some embodiments, the outer layer is provided over the reinforcing layer, such as to enclose or encapsulate the reinforcing layer within the wall of the reinforced flexible tubular elongate member. The outer layer may fill voids or gaps in the reinforcing layer, such as to embed the reinforcing layer within the outer layer and/or to form a sandwich-like structure comprising the inner layer, the reinforcing layer, and the outer layer.

The reinforcing layer may be formed, shaped, and configured in any of a desired configurations to provide or impart a desired property to the flexible tubular elongate member along the length thereof (extended along a longitudinal axis thereof) and/or along a portion of the circumference of the flexible tubular elongate member (about the longitudinal axis thereof). In some embodiments, the reinforcing layer is formed of a plurality of elongated elements (filaments, fibers, strands, ribbons, etc., referenced herein as filaments for the sake of convenience and without intent to limit), such as wires, which extend longitudinally along the length of the flexible tubular elongate member. The reinforcement filaments of the reinforcing layer may be formed of any of a desired materials, such as a metal, a metal alloy (stainless steel, a nickel-titanium alloy or other nickel alloy, aa tungsten alloy, a chromium alloy, etc.), a polymer, a metal-polymer composite material, etc., without limitation. The filaments may be formed of a linear elastic or non-super-elastic of super-elastic or shape-memory material, such as selected based on the intended use of the flexible tubular elongate member. The filaments may have any of a variety of cross-sectional shapes (e.g., round, flattened, oblong, etc.), such as to impart a desired characteristic or property to the reinforcing layer. The diameter or thickness of the filaments is selected based on the intended use of the reinforced flexible tubular elongate member to result in a reinforced flexible tubular elongate member with a desired overall diameter as well as a desired wall thickness. At least a portion of the reinforcing layer nay be coated, such as with a polymeric coating material to impart a desired further property or characteristic to one or more filaments of the reinforcing layer. Such coating may be applied in any of a desired manners, such as extrusion (e.g., over each filament), dipping, painting, brush coating, spraying, etc., without intent to limit.

In some embodiments, the filaments (singly or in pairs or in groups of more than two filaments) are braided and/or interwoven and/or intertwined and/or overlapped with other filaments (singly or in pairs or in groups of more than two filaments), in any of a variety of patterns, the specifics of which are not critical to the present disclosure. It will be appreciated that interengagement of the components of the reinforcing layer is not critical to the principles of the present disclosure. In other words, filaments of the reinforcing layer may extend longitudinally along the length of the reinforced flexible tubular elongate member without engaging or otherwise contacting one another, or with only some filaments, but not all filaments, contacting one another. In some embodiments, the reinforcing layer may be a solid-core braid component encapsulated or embedded within material (e.g., polymeric, thermoplastic, etc.) forming the wall of the flexible elongate member.

In various procedures or treatments (such terms being used interchangeably herein without intent to limit), visualization and/or imaging of an anatomical site may be desired or necessary. For instance, to facilitate and/or guarantee proper navigation, deployment, placement, engagement, implantation, etc., of a device (e.g., an implantable device, such as, without limitation, a tissue anchor) and/or system with respect to an anatomical site, a variety of imaging techniques may be used to image devices and/or systems as well as to image the anatomical structures with respect to which the devices and/or systems are positioned. Various imaging techniques utilize various materials (e.g., contrast agents or media used for fluoroscopy, tomography, etc.) during navigation or once at the anatomical site. Such materials may be useful to enhance imaging and/or visualization and/or visibility and/or identification of an anatomical site, such as by increasing the contrast of structure or fluids within the body, delineating borders between tissues, and/or indicating walls or passages of anatomical structures based on the flow of the contrast material with respect to the anatomical structures.

Provision of a flexible tubular elongate member with more than one layer as described above may result in a thicker wall than a single-layer tubular element, further increasing the overall size of the system and limiting the space for provision of additional medium-delivery channels, passages, lumens, etc. (such terms being used interchangeably herein without intent to limit).

Moreover, the use of a reinforcing layer in one or more flexible elongate members of such a system, such as described above, takes up additional space within the wall of such member, thereby increasing the wall thickness of such member, and further increasing the overall size of the system and reducing the available space for a medium-delivery channel to extend through the delivery system. However, elimination of a reinforced flexible tubular elongate member may eliminate one or more desirable characteristics imparted by the reinforced flexible tubular elongate member.

In accordance with various principles of the present disclosure, at least one flexible tubular elongate member used in a transluminal delivery system includes at least one medium-delivery channel within the wall thereof (e.g., between the exterior and the interior of the wall and within the thickness of the wall) without adding further bulk or width to the delivery system. By providing a medium-delivery channel within the wall of the flexible tubular elongate member, an additional medium not previously deliverable with prior such systems may be delivered without impacting the size of the system. Such medium-delivery channel may extend from a proximal end of the flexible tubular elongate member to a distal end of the flexible tubular elongate member to allow a material (e.g., a fluid such as a contrast agent) to be passed from a proximal end of the flexible tubular elongate member (e.g., from a material source, such as with the use of a pump), to a distal end of the flexible tubular elongate member. Additionally or alternatively, such channel may be coupled to a vacuum source to apply vacuum pressure from a proximal end of the flexible tubular elongate member to a distal end of the flexible tubular elongate member positioned, for instance, at a treatment site in need of suction. It will be appreciated that reference herein to a material such as a contrast agent is for the sake of convenience, without intent to limit, and various other mediums may be delivered or withdrawn through channels through flexible tubular elongate members formed in accordance with various principles of the present disclosure.

In some aspects, in accordance with various principles of the present disclosure, at least one longitudinally-extending component of a reinforcing layer of a reinforced flexible tubular elongate member is formed with a medium-delivery channel therein. For instance, a filament of a reinforcing layer of a reinforced flexible tubular elongate member may be a hollow longitudinally-extending filament through which a material may be passed. The use of hollow filaments, such as to form a reinforcing layer, within the wall of a flexible tubular elongate member as described herein allows delivery of a medium through some or all of the filaments of the reinforcing layer. The resulting reinforced flexible tubular elongate member has virtually identical characteristics as a traditionally-formed reinforcing layer with solid core strands, yet also advantageously has the additional ability to deliver a material to an anatomical site through its own generally rigid structure. Furthermore, such configuration and structure negates the need for additional medium delivery devices or alteration of current and future build designs of devices and systems due to size constraints and need for delivery of a medium during a procedure.

In some embodiments, anywhere from one to all of the solid-core braid wires on a braiding machine for forming a braided reinforcing layer for a flexible tubular elongate member maybe replaced with hollow-core braid wires. Such hollow-core braid wires may be braided or otherwise configured with respect to one another with the same setting as used with traditional solid core wires. Once assembly of the flexible tubular elongate member has been completed, the hollow-core braid wires (or the entirety of the wall of the catheter) can deliver a material through the tubular wall of the flexible tubular elongate member in which the braid wires extend, allowing other materials (e.g., devices) to be delivered within the lumen defined by the tubular wall. In some embodiments, the flexible tubular elongate member may be assembled into a delivery/deployment system with the proximal-most portion of the system (e.g., the handle end) in fluid communication with a syringe or pump assembly to supply a material for delivery through the hollow-core braid wires within the wall of the flexible tubular elongate member to the distal-most portion of the system (within the patient). Allowing materials to be delivered through the existing wall of a tubular element negates the need for other devices for delivering materials. Such configuration is especially useful when a procedure is being conducted and the given position of a device (e.g., an implantable device) needs to be accurately known/visualized before deployment of the device. For instance, often times, there is insufficient space/room for an additional independent device for delivering a material such as a contrast agent, and the ability to remove the implantable device in the middle of a procedure (to allow separate contrast agent to enter the system) to check position is not possible or a feasible option.

It will be appreciated that principles of the present disclosure relate to all devices where the position of a device and/or system is important. Medium-delivery channels may be formed within walls of flexible tubular elongate members in accordance with various principles of the present disclosure without affecting the design, operation, configuration, etc., of other aspects of the flexible tubular elongate member or a system in which the flexible tubular elongate member is provided and/or used. For instance, hollow filaments formed in accordance with various principles of the present disclosure to define medium-delivery channels therein are easily integrated into current shaft/catheter designs (e.g., utilizing a braid for reinforcing the shaft composition). Moreover, use of such hollow filaments does not require redesign of the composition of the flexible tubular elongate member other than replacement of anywhere from one to all of the solid braid-wire(s) with hollow braid-wire(s) formed in accordance with various principles of the present disclosure. As such, the present disclosure allows for the delivery of materials such as contrast agents or other solutions through any catheter system where such materials may be desired or required.

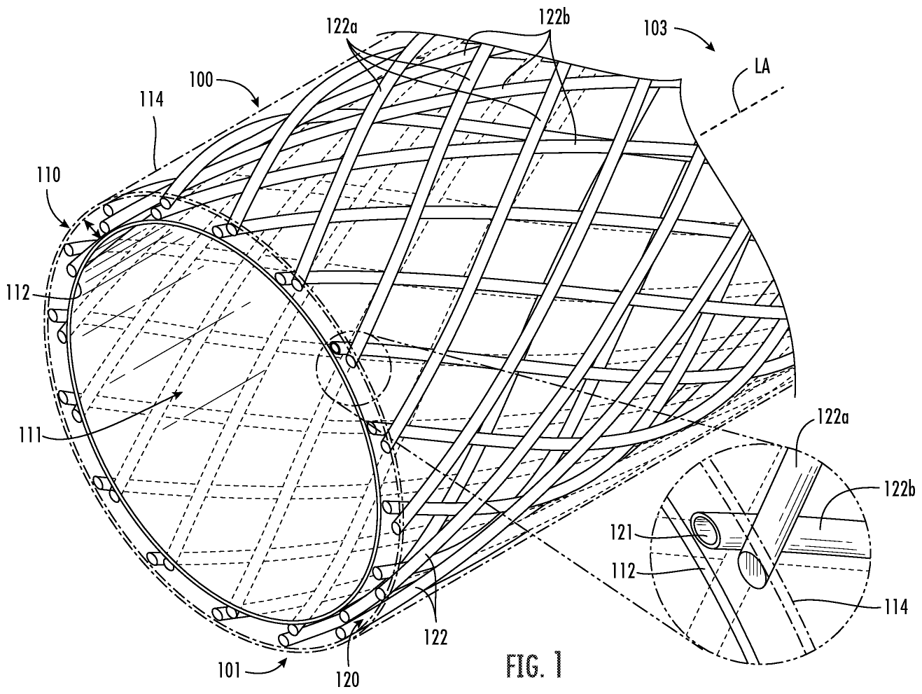

Turning now to the drawings, an example of an embodiment of a flexible tubular elongate member 100 to which principles of the present disclosure may be applied is illustrated in FIG. 1. The illustrated flexible tubular elongate member 100 has a generally tubular wall 110 with a thickness through which one or more medium-delivery channels are formed. In some embodiments, as illustrated in FIG. 1, the flexible tubular elongate member is a reinforced flexible tubular elongate member 100 having a tubular wall 110 formed of an interior wall 112 and an exterior wall 114 (shown in phantom), with a reinforcing layer 120 therebetween. Although the tubular wall 110 of the flexible tubular elongate member 100 defines a working channel 111 extending longitudinally therethrough (along the longitudinal axis LA of the reinforced flexible tubular elongate member 100), a flexible elongate member formed in accordance with various principles of the present disclosure need not have such a working channel. In some embodiments, the interior wall 112 is an inner liner, formed within the reinforcing layer 120 or over which the reinforcing layer 120 is formed. For instance, the inner liner may be provided within the braided catheter, such as to form a lining for the working channel 111, after formation of the braided catheter. The exterior wall 114 extends around, surrounds, encloses, envelops, covers, etc. (such terms being used interchangeably herein without intent to limit) the reinforcing layer 120. In some embodiments, the reinforcing layer 120 is formed over the reinforcing layer 120 and the interior wall 112, such as by extrusion, such as to form an outer jacket or layer covering the reinforcing layer 120. The reinforcing layer 120 may be embedded in one or both of the interior wall 112 or the exterior wall 114. Further details of various manners in which such a reinforced flexible tubular elongate member are made are not critical to principles of the present disclosure and thus not further described herein.

A variety of configurations of reinforcing layers 120 are known in the art, any of which configuration may be used in a reinforced flexible tubular elongate member 100 formed in accordance with various principles of the present disclosure without detracting therefrom. For instance, in the example of an embodiment illustrated in FIG. 1, the reinforcing layer 120 is formed from one or more filaments 122 arranged in any of a variety of manners about the circumference of the reinforced flexible tubular elongate member 100 and extending along the longitudinal axis LA of the reinforced flexible tubular elongate member 100 from a proximal end 101 thereof (not shown, but the general location of which is indicated by reference number 101), to a distal end 103 thereof. In some embodiments, a plurality of filaments 122 forming a reinforcing layer 120 may be interengaged with one another, such as braided, interwoven, intertwined, overlaid, etc., and/or may extend zig-zagging, longitudinally, sinusoidally, helically, etc., singly (spaced apart from one another) or in groups along the longitudinal axis LA of the reinforced flexible tubular elongate member 100. In some embodiments, one or more filaments 122 are coiled to form a reinforcing layer 120. The reinforcing layer 120 illustrated in FIG. 1 has a first plurality of generally helically-extending spaced apart filaments 122a extending around the circumference of the reinforced flexible tubular elongate member 100 in a first direction and layered over a second plurality of generally helically-extending spaced apart filaments 122b extending around the circumference of the reinforced flexible tubular elongate member 100 in an opposite direction. However, any or all of the filaments 122a of the first plurality of filaments 122a may be woven (e.g., under-over) any or all of the filaments 122b of the second plurality of filaments 122b, singly or in groups. For instance, individual or pairs or three or more filaments 122 may weave over-under individual or pairs or three or move filaments 122. The principles of the present disclosure are not limited by a particular pattern of interengagement (or lack of engagement) of filaments 122 forming a reinforcing layer 120. It will be appreciated that there are a variety of additional or alternative acceptable manners of forming a reinforcing layer 120 of a reinforced flexible tubular elongate member 100 in accordance with various principles of the present disclosure.

As described above, provision of a reinforcing layer 120, such as illustrated in FIG. 1, may add to the thickness of the reinforced flexible tubular elongate member 100. In accordance with various principles of the present disclosure, the reinforcing layer 120 serves a dual purpose of reinforcing the flexible tubular elongate member 100 as well as providing medium-delivery channels 121 through the tubular wall 110 of the reinforced flexible tubular elongate member 100. For instance, as illustrated in the detail view of the example of an embodiment of a reinforced flexible tubular elongate member 100 in FIG. 1, one or more of the filaments 122 forming the reinforcing layer 120 may be hollow. The inner diameter of the hollow filaments 122 is selected to allow passage therethrough of a material (e.g., a medium such as a contrast agent) to be delivered to an anatomical site to which the reinforced flexible tubular elongate member 100 is navigated (generally transluminally within the patient's body). It will be appreciated that the dimensions of the hollow filaments 122 may be determined based on a variety of factors, such as the nature of the material to be passed therethrough, the volume of material to be delivered in a given time (including the rate at which the material is to be delivered), the strength of the material forming the filaments 122 (e.g., to maintain the strength of the reinforcing layer 120 while still providing a passage for flow of material therethrough), the overall dimensions of the reinforced flexible tubular elongate member 100, etc. Generally, reinforcing layers formed of a plurality of filaments (e.g., braided, interwoven, etc.) are formed of stainless steel (e.g., 304B stainless steel). To allow formation of a hollow filament 122 in accordance with various principles of the present disclosure, the filaments 122 may be formed of another material, such as nitinol, tungsten, polyamide, polyetheretherketone (PEEK), etc., allowing for a filament to be formed with a sufficiently small outer diameter and with an interior passage therethrough to fit within a tubular element to be navigating within a patient's body. For instance, in some embodiments, one or more filaments 122 of the reinforcing layer 120 may be hollow wires with an outer diameter of approximately 0.007″ (0.0178 mm) and an inner diameter of approximately 0.005″-0.006″ (0.127 mm-0.153 mm), and provided within a flexible tubular elongate member 100 having an outer diameter of approximately 0.1″ (2.54 mm) and an inner diameter of approximately 0.08″ (2.032 mm). It will be appreciated that the dimensions of the filaments 122 may vary widely, depending on the dimensions of the reinforced flexible tubular elongate member 100. For instance, filaments 122 used in a microcatheter may have outer diameters as small as 0.001″ (25.4 μ), whereas filaments 122 used in a large endoscope may have outer diameters as large as about 0.01″ (0.254 mm).

It will further be appreciated that the number of filaments 122 in the reinforcing layer 120 may be selected depending on any of a variety of factors. For instance, the number of filaments 122 may be selected to achieve desired mechanical properties of the reinforcing layer 120, to achieve a desired volumetric need with respect to the material being delivered therethrough (e.g., as determined by the anatomy being imaged, in the case of contrast agents being delivered), the overall size of the reinforced flexible tubular elongate member 100, etc. In some embodiments, a reinforcing layer 120 includes one or more filaments 122 extending longitudinally along the longitudinal axis LA of the reinforced flexible tubular elongate member 100. For instance, in some embodiments, a plurality of filaments 122, including two to as many as thirty-two (32) filaments 122 or even as many as sixty-four (64) filaments 122 (including any number of filaments therebetween, or even more) may form a reinforcing layer 120 of a flexible tubular elongate member 100 formed in accordance with various principles of the present disclosure, with one or more of those filaments 122 being hollow with a medium-delivery channel 121 defined therethrough.

Devices, systems, and methods of the present disclosure may be used alone or together with other devices, systems, and methods. It will be appreciated that there are a variety of applications for a reinforced flexible tubular elongate member 100 formed in accordance with various principles of the present disclosure. For instance, principles of the present disclosure may be applied in devices and systems with one or more flexible tubular elongate members used in a variety of transluminal procedures. As discussed above, the dimensions (e.g., width or diameter) of devices and systems which are navigated within the body are generally constrained by the size and shape and contours of the anatomical structures through and which the devices and systems are navigated, leaving no additional space for medium-delivery channels. Application of principles of the present disclosure to at least one flexible elongate member of a system with more than one flexible elongate member provides the ability to deliver mediums (such as contrast agent) through components already being used in the system for other devices or systems. For instance, a catheter system may be modified to have one or more channels defined within walls of at least one of the catheters of the system. More particularly, a reinforced flexible tubular elongate member with filaments forming the reinforcing layer thereof may be modified in accordance with various principles of the present disclosure to define medium-delivery channels through one or more of the filaments of the reinforcing layer to create pathways for delivering mediums therethrough. As such, elements typically already present and used in the system (e.g., reinforcing filaments such as braid-wires, or the space in the wall itself) are utilized to deliver materials through the system, thereby negating the need for an entirely separate and independent catheter assembly for delivering such material (as typically required in prior art systems).

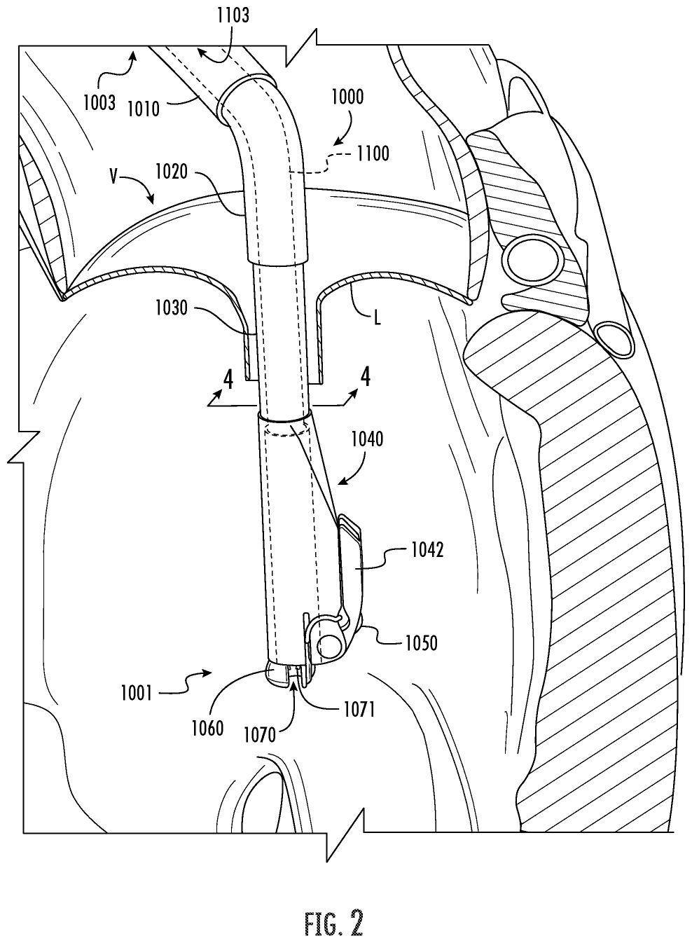

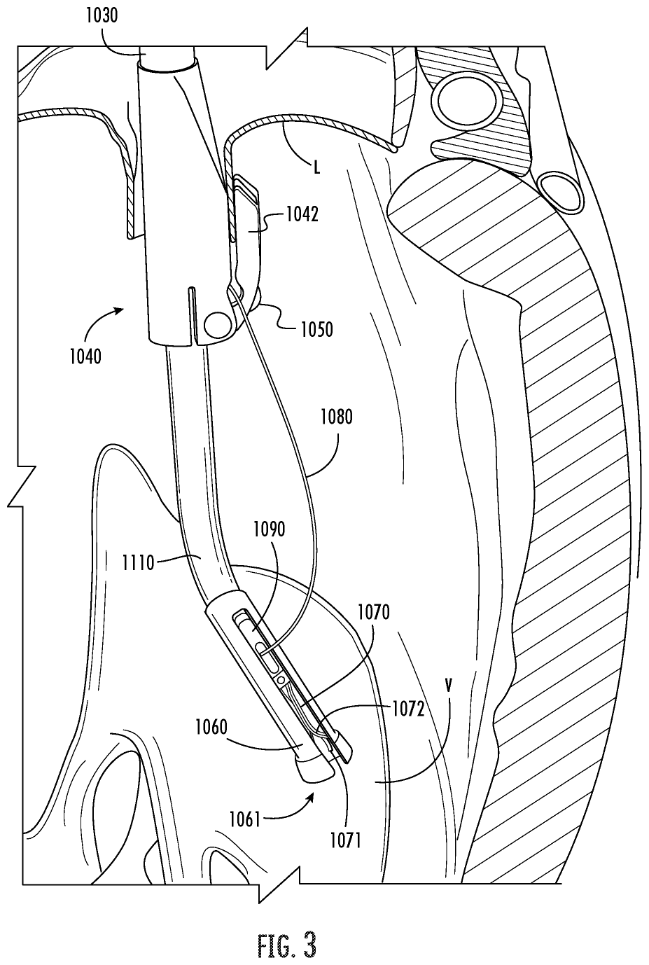

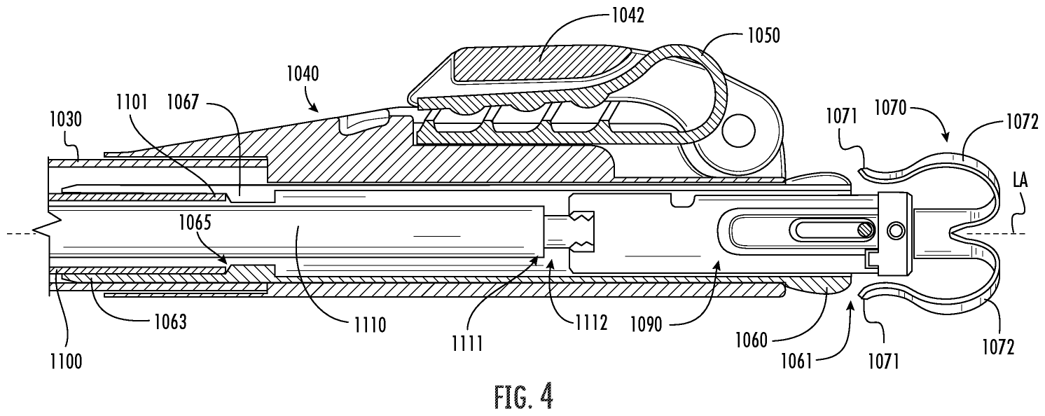

An example of an embodiment of a system and device to which various principles of the present disclosure may be applied is a delivery and deployment system 1000 with multiple “stacked” flexible tubular elongate members (positioned one within the other coaxially and/or coextensively within another flexible tubular elongate member), such as illustrated in FIG. 2, FIG. 3, and FIG. 4. It will be appreciated that reference is made to delivery and deployment, but a system incorporating principles of the present disclosure need not be limited to either or both such purposes. The various elongate members of the system 1000 may include a delivery guide sheath 1010, a delivery catheter 1020, a grasper shaft 1030, an anchor garage catheter 1100, and a stylet 1110, any or all of which may be flexible elongate elements (e.g., catheter, sheath, shaft, tube, etc.) steerable through tortuous pathways through the body to allow for transluminal (e.g., transcatheter, in contrast with open surgery) delivery of medical devices within the body, thereby avoiding invasive, open surgery. In the illustrated example, at least one of the flexible elongate members may be a reinforced flexible tubular elongate member formed in accordance with various principles of the present disclosure to deliver a material to the distal end 1001 of the system, such as from a source at a proximal end 1003 (indicated generally but not precisely shown) of the system 1000.

The illustrated example of a delivery and deployment system 1000 is configured to deliver and to deploy devices and systems for cardiac procedures such as heart valve repair (e.g., to ensure proper functioning and closure of heart valves). More particularly, the illustrated devices and systems are configured for repositioning, repairing, and/or replacing one or more heart valve leaflets and/or chordae tendineae. However, it will be appreciated that principles of the present disclosure may be applied to other devices and systems without limitation, particularly to other devices and systems delivered transluminally/transcatheterally.

In various procedures, such as mitral valve therapies in which a device is implanted with respect to heart tissue, there is a need for the ability to identify if a device is in perfect contact with the tissue for proper engagement with the tissue. Some devices must be completely in contact with tissue before deployment to ensure proper penetration and implantation into tissue. For instance, devices such as tissue anchors may be deployed incorrectly if not initially in complete contact with tissue, and may thereby compromise the strength of anchor implant. One manner of facilitating identification of the position of the device and the anatomical structure is to use an imaging technique in conjunction with a contrast agent. In some instances, the position of a component at one of the innermost, if not the innermost, flexible elongate members of the system must be determined accurately. However, the inner flexible elongate members of a stacked system affords very little room for a delivery channel for the contrast medium. Principles of the present disclosure are applied to at least one of the flexible elongate members to allow a contrast medium to be delivered to facilitate imaging and placement of a component/device to achieve secure positioning of the component and/or implanting of a device, as will now be described in further detail.

The example of an embodiment of a delivery and deployment system 1000 illustrated in FIG. 1 may be delivered to an anatomical site (in this example, a heart ventricle) by a delivery guide sheath 1010. The delivery guide sheath 1010 may be introduced into the body with a dilator (not shown, but which may be any known dilator) to extend transluminally to the heart, such as through the femoral artery to cross through the septal wall into the ventricle. A delivery catheter 1020 extends within and through the delivery guide sheath 1010 to deliver one or more devices to an anatomical site, such as the heart. In the illustrated example, a grasper shaft 1030 extends through the delivery catheter 1020 to deliver a device in the form of a leaflet clip spreader 1040 configured to deliver a leaflet clip 1050 to a heart leaflet L. The leaflet clip 1050 is shown with the spreader arm 1042 of the leaflet clip spreader 1040 clamping the leaflet clip 1050 on a leaflet L in FIG. 3, with the leaflet clip 1050 being shown more clearly in FIG. 4. Additionally, in the illustrated example of an embodiment, a delivery device in the form of an anchor garage 1060 is delivered within and through the leaflet clip spreader 1040 to facilitate delivery and deployment of a deployable device, such as an anchor 1070, into the heart (e.g., into cardiac tissue such as papillary muscle tissue). The anchor garage 1060 may define a lumen therein in which the anchor 1070 may be delivered. The anchor garage 1060 may be extended distally out of the distal end 1041 of the leaflet clip spreader 1040, as shown, for example, in FIG. 2, to be projected to a desired anatomical site for implanting the anchor 1070. The anchor 1070 may then be extended out the distal end 1061 of the anchor garage 1060 and into tissue to secure an artificial chordae tendineae 1080 (e.g., an expanded polytetrafluoroethylene (ePTFE) suture), extending from the leaflet clip 1050, to the cardiac tissue of the ventricle (e.g., to papillary muscle tissue) to restore proper functioning of the leaflet L. An artificial chordae tendineae tensioning and locking device 1090 may be used to set the tension on the artificial chordae tendineae 1080 as desired, indicated, necessary, etc.

The anchor garage 1060 is delivered on the distal end 1101 of a generally-flexible tubular elongate anchor garage catheter 1100, shown in phantom in FIG. 2 extending through the grasper shaft 1030, and shown in FIG. 3 extending distally out of the distal end 1041 of the leaflet clip spreader 1040. As illustrated in cross-section in FIG. 4, the distal end 1101 of the anchor garage catheter 1100 may be coupled to the proximal end 1063 of the anchor garage 1060 such as by being fitted within a counterbore 1065 formed within the proximal end 1063 of the anchor garage 1060. The anchor 1070 (with the artificial chordae tendineae tensioning and locking device 1090 coupled thereto, such as coupled to a proximal end of the anchor 1070) is delivered at the distal end 1111 of a stylet 1110 (as shown in FIG. 4). The stylet 1110 and the anchor 1070 may be coupled together, such as via a coupling 1112 between the stylet 1110 and the artificial chordae tendineae tensioning and locking device 1090, in a manner allowing deployment of the anchor 1070 from the anchor garage 1060 and adjustment of tension on the artificial chordae tendineae 1080 via the artificial chordae tendineae tensioning and locking device 1090.

It will be appreciated that details of structures and functions of the leaflet clip

spreader 1040, the leaflet clip 1050, the anchor garage 1060, the anchor 1070, the artificial chordae tendineae 1080, and the artificial chordae tendineae tensioning and locking device 1090 do not form a part of the present disclosure, and thus are not discussed or described in further detail herein. Detailed descriptions of examples of such devices and systems may be found, for example, in U.S. Patent Application Publication US2021/0007847, titled DEVICES, SYSTEMS, AND METHODS FOR CLAMPING A LEAFLET OF A HEART VALVE, and published on Jan. 14, 2021; U.S. Patent Application Publication US2021/0000597, titled DEVICES, SYSTEMS, AND METHODS FOR ADJUSTABLY TENSIONING AN ARTIFICIAL CHORDAE TENDINEAE BETWEEN A LEAFLET AND A PAPILLARY MUSCLE OR HEART WALL, and published on Jan. 7, 2021; U.S. Patent Application Publication US2021/0000599, titled DEVICES, SYSTEMS, AND METHODS FOR ARTIFICIAL CHORDAE TENDINEAE, and published on Jan. 7, 2021; U.S. Patent Application Publication US2021/0000598, titled DEVICES, SYSTEMS, AND METHODS FOR ANCHORING AN ARTIFICIAL CHORDAE TENDINEAE TO A PAPILLARY MUSCLE OR HEART WALL, and published on Jan. 7, 2021; U.S. Patent Application Publication US 2022/0096235, titled DEVICES, SYSTEMS, AND METHODS FOR ADJUSTABLY TENSIONING ARTIFICIAL CHORDAE TENDINEAE IN A HEART, and published on Mar. 31, 2022; U.S. Patent Application Publication US 2023/0123832, titled DEVICES, SYSTEMS, AND METHODS FOR CLAMPING A LEAFLET OF A HEART VALVE, and published on Apr. 20, 2023; U.S. Patent Application Publication US 2023/0062599, titled DEVICES, SYSTEMS, AND METHODS FOR ANCHORING AN ARTIFICIAL CHORDAE TENDINEAE TO CARDIAC TISSUE, and published on Mar. 2, 2023; U.S. Patent Application Publication US 2023/0149170, titled DEVICES, SYSTEMS, AND METHODS FOR POSITIONING A LEAFLET CLIP, and published May 18, 2023; and U.S. provisional patent application ___/______ [Attorney Docket No. 2002.2715100, formerly Attorney Docket No. 8150.0817Z], titled DEVICES, SYSTEMS, AND METHODS FOR POSITIONING AN ANCHOR FOR AN ARTIFICIAL CHORDAE TENDINEAE, and filed Dec. 20, 2021, all of which applications are hereby incorporated by reference herein in their entireties and for all purposes.

It will be appreciated that the illustration of the position of the anchor 1070 in FIG. 4 as extending out the distal end 1061 of the anchor garage 1060 while the anchor garage 1060 is within the leaflet clip 1050 is for the sake of convenience as a compact depiction of the various stacked components along with a depiction of a configuration of the talons 1072 of the anchor 1070 when deployed. When the anchor garage 1060 is within the leaflet clip spreader 1040, as depicted in FIG. 2 and FIG. 4, the anchor 1070 is typically in a generally compact delivery configuration within the anchor garage 1060, facilitating delivery through narrow body passages, as illustrated in FIG. 3, and not in a deployed configuration as illustrated in FIG. 4. Generally, to deploy the anchor 1070, the anchor garage 1060 is distally extended out from the leaflet clip spreader 1040, and beyond the distal end 1041 of the leaflet clip spreader 1040, such as illustrated in FIG. 3. Once the anchor garage 1060 is in a position such as illustrated in FIG. 3, the anchor garage 1060 may be placed into contact with tissue in which an anchor 1070 is to be implanted, and the anchor 1070 may then be pushed distally out of the anchor garage 1060 and into tissue. The anchor garage 130 may have a blunt open distal end 1061 (tip or free end) sized, shaped, configured, and dimensioned to facilitate pushing of the anchor garage 1060 against cardiac tissue to deploy the anchor 1070 out of the anchor garage 1060 and into tissue at the deployment site without potentially pushing the distal end 1061 of the anchor garage 1060 into the cardiac tissue as well.

It is generally desirable for an implantable anchor 1070 to establish a strong purchase in or with the tissue so that the anchor 1070 is not inadvertently withdrawn from the tissue. An anchor 1070 used in accordance with various principles of the present disclosure may include a plurality of talons which may shift from a delivery configuration (e.g., a compact position facilitating delivery through narrow body passages, such as illustrated in FIG. 3) to a deployed configuration for engaging tissue. The distal ends 1071 of the anchor talons 1207 may each be tapered or pointed or otherwise configured to pierce and penetrate tissue when pushed against body tissue. Once within the tissue, the talons 1072 may extend or shift radially outwardly away from the longitudinal axis LA thereof into a deployed configuration to secure the anchor 1070 to the body tissue. For instance, the talons 1072 may curl, curve, bow, bend, etc., such as towards the proximal end of the anchor 1070, as illustrated in FIG. 4.

In some embodiments, tissue anchors 1070 have talons 1072 which are pre-formed or biased (e.g., formed of an elastic and/or shape-memory material such as Nitinol and/or heat-treated) to shift from a delivery configuration to a deployed configuration upon being extended distally out of the anchor garage 1060 and without additional forces (other than to push the anchor 1070 out of the anchor garage 1060). In such embodiments, the talons 1072 may be considered to act as a spring to shift the distal ends 1071 into a deployed configuration and are biased to substantially automatically open into the open configuration without external forces moving the anchor talons. To ensure proper deployment of such tissue anchor 1070 and sufficient purchase within tissue, intimate and/or full contact of the talons 1072 with tissue (“proper” contact, generally oriented perpendicular to the surface of the tissue) is necessary at the time of deployment of the anchor 1070. Lack of contact of the anchor with tissue typically will result in that portion of the anchor (e.g., a talon) not purchasing or penetrating the tissue. If the anchor garage 1060 and/or distal ends 1071 of such anchors 1070 are not properly engaged with tissue into which the anchor 1070 is to be implanted, the talons 1072 may move into a deployed configuration without having penetrated the tissue. Accordingly, it is desirable to determine whether the anchor garage 1060 and/or anchor 1070 is in the desired position with respect to tissue to securely implant the anchor 1070 with the tissue.

In accordance with various principles of the present disclosure, the anchor garage catheter 1100 is configured to deliver a material, such as a contrast agent, to the distal end 1061 of the anchor garage 1060 to confirm proper positioning of the anchor garage 1060 and/or anchor 1070 before deployment of the anchor 1070 therefrom. In particular, the anchor garage catheter 1100 may be configured similar to the flexible tubular elongate member 100 described above with respect to FIG. 1 with one or more material-delivery channels defined within the wall of the anchor garage catheter 1100 extending between the proximal end 1103 and the distal end 1101 thereof. In some embodiments, the anchor garage catheter 1100 is a reinforced catheter and the material-delivery channels are defined through one or more filaments forming the reinforcing layer of the wall of the anchor garage catheter 1100. Proximal ends of the material-delivery channels at the proximal end 1101 of the anchor garage catheter 1100 (not shown, but generally indicated in FIG. 2) may be coupled with a source of a material to be delivered through the channels within the anchor garage catheter 1100 (such as with the assistance of a pump such as known to those of ordinary skill in the art). The distal ends of such material-delivery channels open at the distal end 1101 of the anchor garage catheter 1100 to deliver material into the interior of the anchor garage 1060. As illustrated in FIG. 4, a groove 1067 may be formed in the counterbore 1065 in the anchor garage 1060 within which the distal end 1101 of the anchor garage catheter 1100 is seated to allow material to flow from the medium-delivery channels within the wall of the anchor garage catheter 1100 into the interior of the anchor garage 1060 (e.g., within which the anchor 1070 is delivered to the anatomical site). The material delivered by the anchor garage catheter 1100 extends generally axially and distally through the anchor garage 1060 out the distal end 1061 of the anchor garage 1060. The pattern of flow of the material upon exiting the distal end 1061 of the anchor garage 1060 may be viewed with appropriate imaging techniques to determine the position of the distal end 1061 of the anchor garage 1060 with respect to the implantation site for the anchor 1070. As such, proper positioning of the anchor garage 1060 may be achieved to assure proper implantation of the anchor 1070 within the target tissue in which the anchor 1070 is to be implanted.

The direction or pattern of flow of a material such as a contrast agent from the distal end 101 of a reinforced flexible tubular elongate member 100 such as formed in accordance with various principles of the present disclosure may be visualized to indicate the proximity, position, orientation, etc., of the device to be deployed (e.g., the anchor 1070) with respect to the tissue with respect to which the device is to be deployed. As such, a system formed with a flexible tubular elongate member 100 formed in accordance with various principles of the present disclosure allows medical professionals (e.g., surgical team, including any automated system) using the system to implant an anchor 1070 when in contact with tissue or at least when the delivery and deployment device (e.g., anchor garage 1060) sufficiently contacts tissue to ensure proper deployment of the anchor 1070. Such information is conveyed to the medical professionals in any of a variety of manners (e.g., visual or audible indication, such as on a delivery system or separate associated device) to facilitate placement, implantation, etc., of the anchor 1070. For instance, such information may be used to guide placement of the anchor and the deployment and delivery device prior to deployment of the anchor, and/or to determine if repositioning is warranted.

It will be appreciated that principles of the present disclosure are applicable to flexible elongate members used in conjunction with devices and systems other than the examples described herein. As such, although various principles of the present disclosure are described herein with reference to embodiments of an implantable device such as a tissue anchor, and associated devices, systems, and mechanisms, principles of the present disclosure may be applied more broadly to other devices, systems, methods, etc., such as, without limitation, those configured to be positioned with respect to body tissue. The devices, systems, and methods described herein may provide robust solutions for patient safety, with regard to implantable devices or otherwise. Moreover, it will be appreciated that although reference is made herein to delivery of a material, it will be appreciated that withdrawal of a material from an anatomical site, such as by application of suction or vacuum force to the medium-delivery channels described herein, are also within the scope and spirit of the present disclosure.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and / or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A flexible tubular elongate member having a tubular wall defining a lumen through which a medical device is deliverable to an anatomical site, said flexible tubular elongate member comprising at least one hollow filament extending within said tubular wall, from a first end of said flexible tubular elongate member to a second end of said flexible tubular elongate member, and defining a medium-delivery channel therein through which a material may pass within the tubular wall from said first end of said flexible tubular elongate member to said second end of said flexible tubular elongate member.

2. The flexible tubular elongate member of claim 1, wherein said tubular wall is a multi-layer wall.

3. The flexible tubular elongate member of claim 2, wherein said tubular wall comprises an outer layer and an inner layer, with said hollow filament extending therebetween longitudinally along said flexible tubular elongate member.

4. The flexible tubular elongate member of claim 3, wherein said hollow filament is woven with a plurality of reinforcement filaments to form a reinforcing layer between said outer layer and said inner layer of said flexible tubular elongate member.

5. The flexible tubular elongate member of claim 4, wherein more than one of said plurality of reinforcement filaments are hollow, extend from a first end of said flexible tubular elongate member to a second end of said flexible tubular elongate member, and define a medium-delivery channel therein through which a material may pass from said first end of said flexible tubular elongate member to said second end of said flexible tubular elongate member.

6. The flexible tubular elongate member of claim 1, wherein said hollow filament is embedded within the material of said tubular wall.

7. The flexible tubular elongate member of claim 1, wherein said hollow filament is one of a plurality of filaments extending longitudinally along said flexible tubular elongate member and within said tubular wall.

8. The flexible tubular elongate member of claim 7, wherein said plurality of filaments form a reinforcing layer within said tubular wall.

9. The flexible tubular elongate member of claim 8, wherein said hollow filament is one of the plurality of hollow filaments forming said reinforcing layer.

10. A delivery and/or deployment system for delivering and/or deploying a medical device to an anatomical site, said delivery/deployment system comprising:
at least one flexible tubular elongate member having a wall defining a lumen and a medium-delivery channel defined within said wall and extending longitudinally along said flexible tubular elongate member between a proximal end thereof and a distal end thereof; and
a device delivered at said distal end of said flexible tubular elongate member.

11. The delivery/deployment system of claim 10, wherein said device comprises a delivery device having an interior within which a deployable device is positioned.

12. The delivery/deployment system of claim 11, wherein the lumen defined by said flexible tubular elongate member wall is in fluid communication with the interior of said delivery device.

13. The delivery/deployment system of claim 11, wherein said deployable device is a tissue anchor.

14. The delivery/deployment system of claim 13, wherein said tissue anchor is biased to shift from a deployment configuration to a deployed configuration upon exiting an open distal end of said delivery device.

15. The delivery/deployment system of claim 14, wherein a medium is deliverable through the medium-delivery channel within said wall of said flexible tubular elongate member, into the interior of said delivery device, and out the open distal end of said delivery device.

16. The delivery/deployment system of claim 15, wherein said medium-delivery channel is formed within a hollow reinforcement filament extending within the wall of said flexible tubular elongate member between said proximal end and said distal end of said flexible tubular elongate member.

17. A method of delivering a material to a distal end of a reinforced flexible tubular elongate member, said method comprising delivering a material through a medium-delivery channel defined longitudinally along a hollow reinforcement filament extending within the wall of the reinforced flexible tubular elongate member between a proximal end and a distal end of the reinforced flexible tubular elongate member.

18. The method of claim 17, further comprising delivering a material through a plurality of hollow reinforcement filaments braided to form a reinforcing layer within the wall of the reinforced flexible tubular elongate member.

19. The method of claim 17, further comprising delivering a contrast agent to the distal end of the reinforced flexible tubular elongate member through the hollow reinforcement filament to determine the position of the reinforced flexible tubular elongate member with respect to tissue.

20. The method of claim 19, further comprising confirming the position of the distal end of the reinforced flexible tubular elongate member with respect to tissue at an anatomical site, and implanting an implantable device delivered at the distal end of the reinforced flexible tubular elongate member into the tissue.
